(19) United States
(12) Patent Application Publication
(10) Pub. No.: US 2024/0076144 A1
(43) Pub. Date: Mar. 7, 2024

(54) Package Receiving and Storage Apparatus

(71) Applicant: Daifuku Co., Ltd, Osaka-shi (JP)

(72) Inventors: Hiroyuki Fukuda, Osaka-shi (JP); Ryohei Yamauchi, Osaka-shi (JP); Toshitaka Sugano, Osaka-shi (JP); Kimiko Kanbe, Osaka-shi (JP); Tooru Arii, Tokyo (JP); Tooru Siraisi, Tokyo (JP)

(21) Appl. No.: 18/240,108

(22) Filed: Aug. 30, 2023

(30) Foreign Application Priority Data
Sep. 1, 2022 (JP) 2022-139375

(51) Int. Cl. B65G 67/00; A61L 2/22; B65G 1/02; B65G 15/30; B65G 39/10; B65G 65/02

(52) U.S. Cl. CPC B65G 67/00; A61L 2/22; B65G 1/02; B65G 15/30; B65G 39/10; B65G 65/02

(57) ABSTRACT

A package receiving and storage apparatus includes a takeoff and landing section; a package receiving section; a storage section; a retrieval section at which the first package stored in the storage section is retrievable; a transport device configured to transport the package between the package receiving section, the storage section, and the retrieval section; and a gas spray device installed at the package receiving section or in a package transport path extending from the package receiving section to the storage section, and configured to spray a gas onto the first package to remove foreign matter adhering to the first package.

### CROSS-REFERENCE TO RELATED APPLICATIONS

This application claims priority to Japanese Patent Application No. 2022-139375 filed Sep. 1, 2022, the disclosure of which is hereby incorporated by reference in its entirety.

### BACKGROUND OF THE INVENTION

### 1. Field of the Invention

The present invention relates to a package receiving and storage apparatus that receives a package from an unmanned aircraft.

### 2. Description of Related Art

Drone ports that receive packages from unmanned aircraft are known. Patent Document 1 (JP 2021-046111A) discloses a drone port that includes a takeoff and landing section on an upper surface of a framework of a building, and receives a package released from an unmanned aircraft that has landed on the takeoff and landing section.

The takeoff and landing section of the drone port described in Patent Document 1 is open to the outside, and therefore foreign matter such as water droplets and dust may adhere to a package when the package is released and transported at the takeoff and landing section. Depending on the structure of the unmanned aircraft, foreign matter such as water droplets and dust may also adhere to the package during flight. In the case where a package that has been released from the unmanned aircraft is stored in a storage section, the foreign matter such as water droplets and dust adhering to the package may separate from the package inside the storage section and accumulate as waste matter, dirtiness, or the like.

### SUMMARY OF THE INVENTION

Therefore, in view of the foregoing, there is a desire to realize a technique that makes it possible to easily avoid the case where foreign matter adhering to a package accumulates as waste matter, dirtiness, or the like in a storage section.

A package receiving and storage apparatus according to the present disclosure includes: a takeoff and landing section at which an unmanned aircraft is capable of taking off and landing; a package receiving section configured to receive a first package from the unmanned aircraft; a storage section configured to store a plurality of packages including the first package; a retrieval section at which the first package stored in the storage section is retrievable; a transport device configured to transport the first package between the package receiving section, the storage section, and the retrieval section; and a gas spray device installed at the package receiving section or in a package transport path extending from the package receiving section to the storage section, and configured to spray a gas onto the first package to remove foreign matter adhering to the first package.

With this configuration, it is possible to remove foreign matter adhering to the package received from the unmanned aircraft before storing the package in the storage section. Accordingly, it is possible to easily avoid, for example, a situation where foreign matter separated from the package inside the storage section accumulates as waste matter, dirtiness, or the like inside the storage section. Note that such foreign matter is assumed to be, for example, water droplets resulting from rain and snow, dust, fallen leaves, and the like. Also, this configuration can reduce the possibility that a package having foreign matter adhering thereto arrives at the retrieval section.

Further features and advantages of the present disclosure will become apparent from the following description of illustrative and non-limiting embodiments with reference to the drawings.

### BRIEF DESCRIPTION OF THE DRAWINGS

FIG. 1 is a side view of a package receiving and storage apparatus according to a first embodiment.

FIG. 2 is a plan view showing the package receiving and storage apparatus on a roof in FIG. 1.

FIG. 3 is a plan view showing the package receiving and storage apparatus on a second floor in FIG. 1.

FIG. 4 is a plan view showing the package receiving and storage apparatus on a first floor in FIG. 1.

FIG. 5 is a plan view showing a package receiving and storage apparatus according to a second embodiment.

FIG. 6 is a plan view showing a package receiving and storage apparatus according to a third embodiment.

FIG. 7 is a side view showing the package receiving and storage apparatus according to the third embodiment.

FIG. 8 is a plan view showing a package receiving and storage apparatus according to a fourth embodiment.

FIG. 9 is a side view showing the package receiving and storage apparatus according to the fourth embodiment.

### DESCRIPTION OF THE INVENTION

### First Embodiment

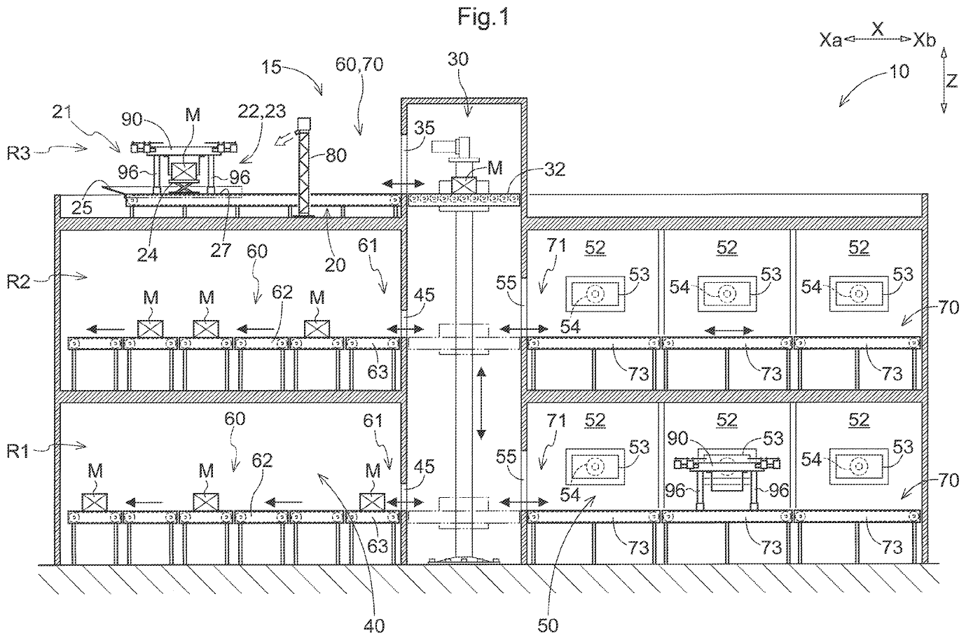

A package receiving and storage apparatus 10 according to the present embodiment will be described with reference to the drawings. FIG. 1 is a cross-sectional view showing a configuration of the package receiving and storage apparatus 10. The package receiving and storage apparatus 10 includes a takeoff and landing section 21 where an unmanned aircraft 90 takes off and lands, and a package receiving section 22 that receives at least one package M from the unmanned aircraft 90. Also, the package receiving and storage apparatus 10 includes a storage section 40 in which a plurality of packages M are stored, and retrieval sections 45 at which the package M stored in the storage section 40 are retrievable. Also, the package receiving and storage apparatus 10 includes a transport device 15 that transports the package M between the package receiving section 22, the storage section 40, and the retrieval sections 45.

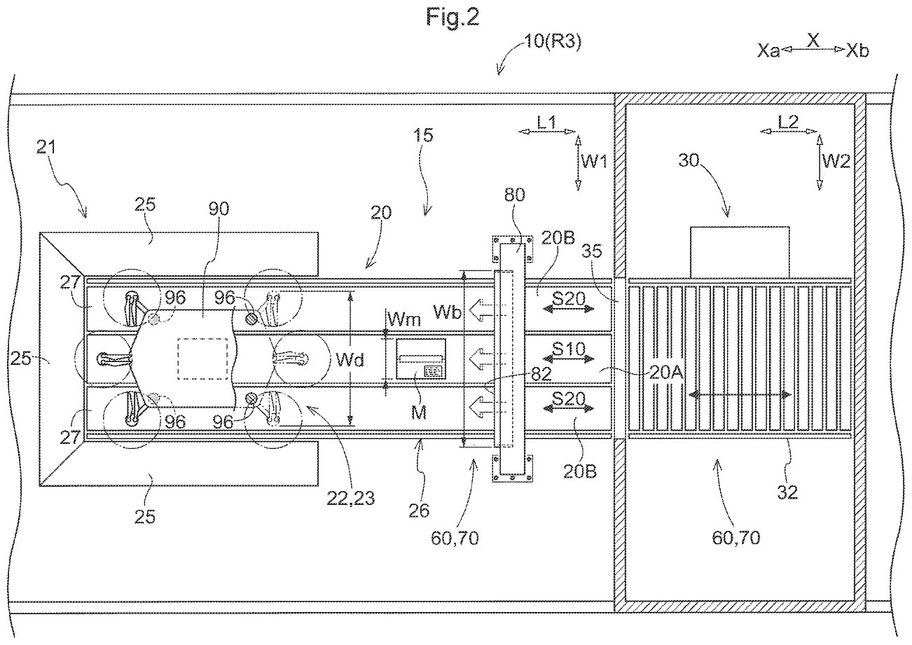

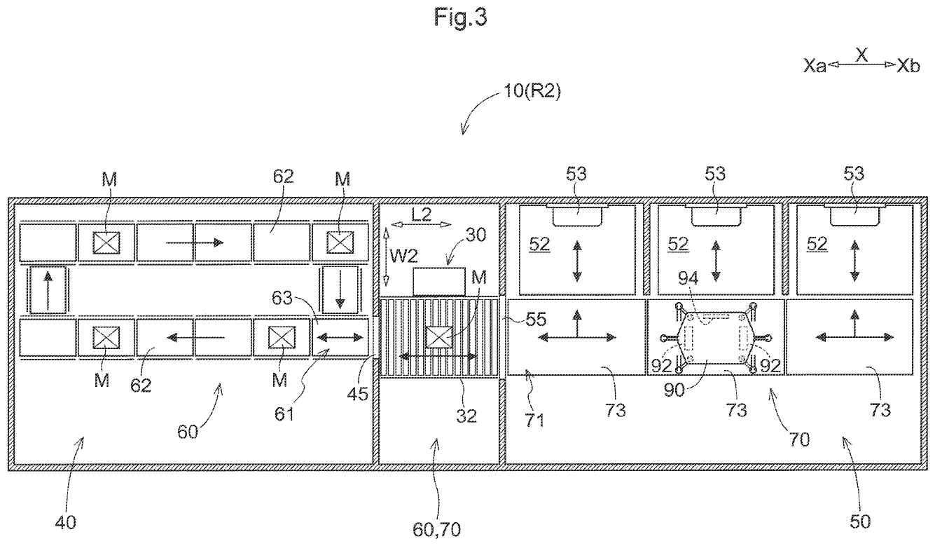

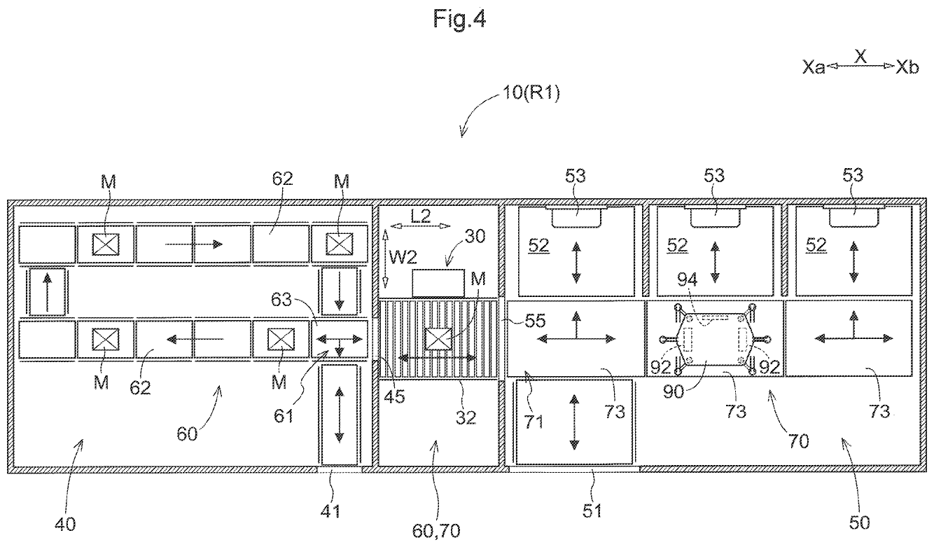

In the present embodiment, the package receiving and storage apparatus 10 is provided in one building. FIG. 2 is an enlarged plan view showing the package receiving and storage apparatus 10 on a roof R3. FIG. 3 is a plan view showing the package receiving and storage apparatus 10 on a second floor R2. FIG. 4 is a plan view showing the package receiving and storage apparatus 10 on a first floor R1. In the present embodiment, the package receiving and storage apparatus 10 includes a package delivery section 23 that delivers the package M to the unmanned aircraft 90. In the present embodiment, the package receiving and storage apparatus 10 includes a replenishment housing section 50 that houses a plurality of unmanned aircraft 90 and replenishes energy (specifically, the energy required for flight) to the housed unmanned aircraft 90.

Each unmanned aircraft 90 is, for example, a fixed-wing aircraft or a rotary-wing aircraft that is remotely operable or capable of autonomous flight, for example. In the present embodiment, the unmanned aircraft 90 is an electrically powered rotary-wing aircraft capable of vertical takeoff and landing. In the example shown in FIG. 3, the unmanned aircraft 90 is rechargeable, and includes storage batteries 92. Also, the unmanned aircraft 90 includes a power receiving coil 94. The unmanned aircraft 90 is a multicopter (so-called drone) capable of autonomous flight. As shown in FIGS. 1 and 2, the unmanned aircraft 90 includes ground contact parts 96. In the example shown in FIG. 2, the ground contact parts 96 are constituted by four leg parts. However, the ground contact parts 96 may be constituted by skids or wheels.

In the present embodiment, the transport device 15 includes an external transport device 20 that is disposed above the storage section 40 and transports the package M at least in the horizontal direction, and a lifter 30 that transports the package M at least in a vertical direction Z. In the present embodiment, the external transport device 20 transports the unmanned aircraft 90 between the takeoff and landing section 21 and the lifter 30. Also, a first entrance 35 is disposed between the external transport device 20 and the lifter 30. Here, the vertical direction Z is a direction extending in a vertical direction. A specific direction X is a specific direction extending in the horizontal direction, a first side Xa in the specific direction X is one side in the specific direction, and a second side Xb in the specific direction X is the other side in the specific direction. As shown in FIG. 2, a transport direction L1 is a direction extending along a transport path realized by the external transport device 20, and a transport width direction W1 is a direction extending orthogonal to the transport direction L1 in a vertical view. Examples of the external transport device 20 include a roller conveyor and a belt conveyor. The external transport device 20 corresponds to a first transporter.

In the present embodiment, the lifter 30 transports both the package M and the unmanned aircraft 90 at least in the vertical direction Z. The storage section 40 is adjacent to the lifter 30 on the first side Xa in the specific direction. The replenishment housing section 50 is adjacent to the lifter 30 on the second side Xb in the specific direction. In the example shown in FIG. 2, the lifter 30 includes an internal transport device 32 capable of transporting the package M and the unmanned aircraft 90 in the specific direction X. Here, a transport direction L2 is a direction extending along a transport path realized by the internal transport device 32, and a transport width direction W2 is a direction extending orthogonal to the transport direction L2 in a vertical view. Examples of the internal transport device 32 include a roller conveyor and a belt conveyor. The lifter 30 corresponds to a second transporter.

In the present embodiment, the takeoff and landing section 21 includes support stands 27 that support the unmanned aircraft 90 from below. The support stands 27 are disposed above the storage section 40 and the replenishment housing section 50. In the illustrated example, the takeoff and landing section 21 is provided on the roof R3 of the building. Also, the takeoff and landing section 21 is intended for vertical takeoff and landing. Preferably, the package receiving and storage apparatus 10 includes an opening/closing roof that covers the takeoff and landing section 21. Preferably, the takeoff and landing section 21 includes a heater for melting frozen water on the takeoff and landing section 21.

In the present embodiment, the takeoff and landing section 21 overlaps the storage section 40 in a vertical view. In the example shown in FIG. 2, the external transport device 20 also serves as the takeoff and landing section 21. The takeoff and landing section 21 includes, on at least one side thereof in the transport width direction W1, an inclined section 25 inclined upward in the vertical direction Z in a direction away from the external transport device 20. The inclined section 25 can facilitate the vertical takeoff and landing of the unmanned aircraft 90 on the external transport device 20, thus making the dimension of the external transport device 20 in the transport width direction W1 relatively smaller.

In the present embodiment, the storage section 40 is disposed below the support stands 27. Also, the storage section 40 is disposed inside the building. The replenishment housing section 50 is disposed inside the building. The package receiving section 22 and the package delivery section 23 are disposed on the roof R3 of the building. In the example shown in FIG. 4, the package receiving and storage apparatus 10 includes a package entrance/exit port 41 and an unmanned aircraft entrance/exit port 51 on the first floor R1 of the building. The package entrance/exit port 41 is used for, for example, storing and retrieving the package M without using the unmanned aircraft 90. The unmanned aircraft entrance/exit port 51 is used for, for example, performing maintenance and inspection of the unmanned aircraft 90. In the example shown in FIG. 1, the package receiving and storage apparatus 10 is provided in a warehouse having two above-ground floors, and the first floor R1 of the warehouse is the first above-ground floor. Note that the storage section 40 and the replenishment housing section 50 of the package receiving and storage apparatus 10 are preferably automatic warehouses. Here, an automatic warehouse refers to a warehouse configured to automatically store and retrieve storage objects.

In the present embodiment, the package delivery section 23 includes a package delivery device 24 that delivers the package M to the unmanned aircraft 90. In the present embodiment, the package delivery section 23 also serves as the package receiving section 22, and the package delivery device 24 also serves as a package receiving device that separates the package M from the unmanned aircraft 90. Examples of the package delivery device 24 include a package receiving robot and an elevator. In the present embodiment, the external transport device 20 also serves as the package receiving section 22. In the present embodiment, the external transport device 20 also serves as the package delivery section 23.

In the present embodiment, the external transport device 20 is disposed higher than the storage section 40 and the replenishment housing section 50 in the vertical direction Z. Also, the external transport device 20 overlaps the storage section 40 in a vertical view taken in the vertical direction Z. The external transport device 20 also serves as the package receiving section 22, and transports the package M to the lifter 30 from a position directly below the unmanned aircraft 90 supported by the support stands 27. The external transport device 20 is configured to execute a package transport operation S10 of transporting a package M separated from an unmanned aircraft 90 to the lifter 30 in the horizontal direction, and an aircraft transport operation S20 of transporting the unmanned aircraft 90 that released the package M and landed on the external transport device 20 to the lifter 30 in the horizontal direction.

In the present embodiment, as shown in FIG. 2, the external transport device 20 includes a distribution transport device 26 configured to, in response to an unmanned aircraft 90 and a package M separated from the unmanned aircraft 90 being placed on an upper surface of the external transport device 20, transport the package M and the unmanned aircraft 90 separately. In the present embodiment, the aircraft transport operation S20 corresponds to an operation in which the external transport device 20 transports only the unmanned aircraft 90 using the distribution transport device 26, and the package transport operation S10 corresponds to an operation in which the external transport device 20 transports only the package M using the distribution transport device 26.

In the present embodiment, the external transport device 20 includes aircraft transport surfaces 20B that support and transport the ground contact parts 96 of the unmanned aircraft 90, and a package transport surface 20A that supports and transports the package M, and the transport speeds of the aircraft transport surface 20B and the package transport surface 20A can be set different from each other. In the present embodiment, the package transport surface 20A and the aircraft transport surfaces 20B function as the distribution transport device 26. In the present embodiment, the aircraft transport surfaces 20B function as the support stands 27. In the illustrated example, the aircraft transport surfaces 20B are provided on both outer sides of the package transport surface 20A in the transport width direction W1.

The package receiving and storage apparatus 10 includes a package transport device 60 that transports the package M between the package receiving section 22 and the storage section 40, and between the package delivery section 23 and the storage section 40. Also, the package receiving and storage apparatus 10 includes an aircraft transport device 70 that transports the unmanned aircraft 90 between the takeoff and landing section 21 and the replenishment housing section 50. In the present embodiment, the package transport device 60 and the aircraft transport device 70 include a shared external transport device 20. In the present embodiment, the package transport device 60 and the aircraft transport device 70 include a shared lifter 30.

In the present embodiment, as shown in FIG. 3, the package transport device 60 includes the external transport device 20, the lifter 30, and a storage transport device 61 that transports the package M between the lifter 30 and the storage section 40. In the present embodiment, the above-described internal transport device 32 included in the lifter 30 transports a package M toward the first side Xa in the specific direction, whereby the package M is transported to the storage section 40. In the illustrated example, a loop transport device 62 and a branch transport device 63 are provided inside the storage section 40. A package M that has been transported from the lifter 30 to the storage section 40 is placed on the loop transport device 62 for storage. The branch transport device 63 transports the package M to either the loop transport device 62 or the lifter 30. In the example shown in FIGS. 3 and 4, the loop transport device 62 and the branch transport device 63 are provided on each floor, and the branch transport device 63 provided on the first floor R1 transports the package M to the loop transport device 62, the lifter 30, or the package entrance/exit port 41. In the illustrated example, the internal transport device 32 and the branch transport device 63 function as the storage transport device 61 that transports the package M between the lifter 30 and the storage section 40.

In the present embodiment, the storage section 40 is installed spanning a plurality of floors of the building. In the present embodiment, second entrances that function as the above-described retrieval sections 45 are disposed between the storage section 40 and the lifter 30. The storage section 40 includes, inside the storage section 40, the storage transport device 61 capable of storing and transporting the package M, and thus functions as an automatic warehouse of for packages M.

In the present embodiment, as shown in FIG. 3, the aircraft transport device 70 includes a housing transport device 71 that transports the unmanned aircraft 90 between the external transport device 20, the lifter 30, and the replenishment housing section 50. In the present embodiment, the above-described internal transport device 32 included in the lifter 30 transports an unmanned aircraft 90 toward the second side Xb in the specific direction, whereby the unmanned aircraft 90 is transported to the replenishment housing section 50. In the illustrated example, the branch transport device 73 is provided inside the replenishment housing section 50. An unmanned aircraft 90 that has been transported from the lifter 30 to the replenishment housing section 50 is transported to either a hangar 52 described below or the lifter 30 by the branch transport device 73. In the example shown in FIGS. 3 and 4, the branch transport device 73 is provided on each floor, and the housing transport device 71 provided on the first floor R1 is configured to transport the unmanned aircraft 90 to the unmanned aircraft entrance/exit port 51. In the illustrated example, the internal transport device 32 and the branch transport device 73 function as the housing transport device 71 that transport the unmanned aircraft 90 between the lifter 30 and the replenishment housing section 50.

In the present embodiment, the replenishment housing section 50 is installed spanning a plurality of floors of the building. In the illustrated example, third entrances 55 are disposed between the replenishment housing section 50 and the lifter 30. The replenishment housing section 50 includes the housing transport device 71 capable of transporting the unmanned aircraft 90 inside the replenishment housing section 50, and thus functions as an automatic warehouse of the unmanned aircraft 90.

In the present embodiment, the replenishment housing section 50 includes a plurality of hangars 52. The hangars 52 each include a wireless charging device 53. Each charging device 53 includes a power feeding coil 54 and supplies power to the storage battery 92 via the power receiving coil 94 of an unmanned aircraft 90 housed in the hangar 52. This enables the replenishment housing section 50 to wirelessly charge the unmanned aircraft 90 housed therein. In this manner, in the present embodiment, the replenishment of energy to the unmanned aircraft 90 by the replenishment housing section 50 is performed by charging the storage battery 92. In the illustrated example, a floor portion of the hangar 52 can transport the unmanned aircraft 90 to and from the branch transport device 73.

Referring back to FIG. 2, the package receiving and storage apparatus 10 includes a gas spray device 80 that is installed in a transport path of the package M extending from the package receiving section 22 to the storage section 40, and that sprays a gas onto the package M to remove foreign matter adhering to the package M. In the present embodiment, the gas spray device 80 is installed in such a manner as to spray the gas onto the package M located on the external transport device 20. In the illustrated example, the gas spray device 80 is shaped like a gate, and the package M is transported directly below the gas spray device 80. For example, air, nitrogen, carbon dioxide, dimethyl ether, propane, or the like is used as the gas sprayed onto the package M. Preferably, the gas sprayed onto the package M is warmed.

In the present embodiment, the gas spray device 80 is configured to spray the gas at a target position along the transport path realized by the external transport device 20, and spray the gas over a range wider than a dimension Wm of the package M in the transport width direction W1. Also, in the present embodiment, the gas spray device 80 is configured to spray the gas at a target position along the transport path of the external transport device 20, and spray the gap over a range wider than a dimension Wd of the unmanned aircraft 90 in the transport width direction W1. In the illustrated example, the gas spray device 80 includes an upper spray part 82, and a dimension Wb of the upper spray part 82 in the transport width direction W1 is larger than the dimension Wm of the package M and the dimension Wd of the unmanned aircraft 90.

In the present embodiment, the gas spray device 80 is configured to also spray the gas onto an unmanned aircraft 90 landed on the takeoff and landing section 21. In the illustrated example, the gas spray device 80 is disposed on the roof R3 of the building. Preferably, the gas spray device 80 emits the gas only during passage of the package M and the unmanned aircraft 90 therethrough. Furthermore, preferably, the package M and the unmanned aircraft 90 are transported separately, and the gas is sprayed onto both the package M and the unmanned aircraft 90 by the gas spray device 80. The gas may be sprayed onto the unmanned aircraft 90 in a storage mode in which the blades of the unmanned aircraft 90 are folded, or in a flight mode in which the blades the unmanned aircraft 90 are unfolded. In the illustrated example, the dimension Wd of the unmanned aircraft 90 in the transport width direction W1, and a dimension Ld of the unmanned aircraft 90 in the transport direction L1 are dimensions when the unmanned aircraft 90 is in the storage mode.

### Second Embodiment

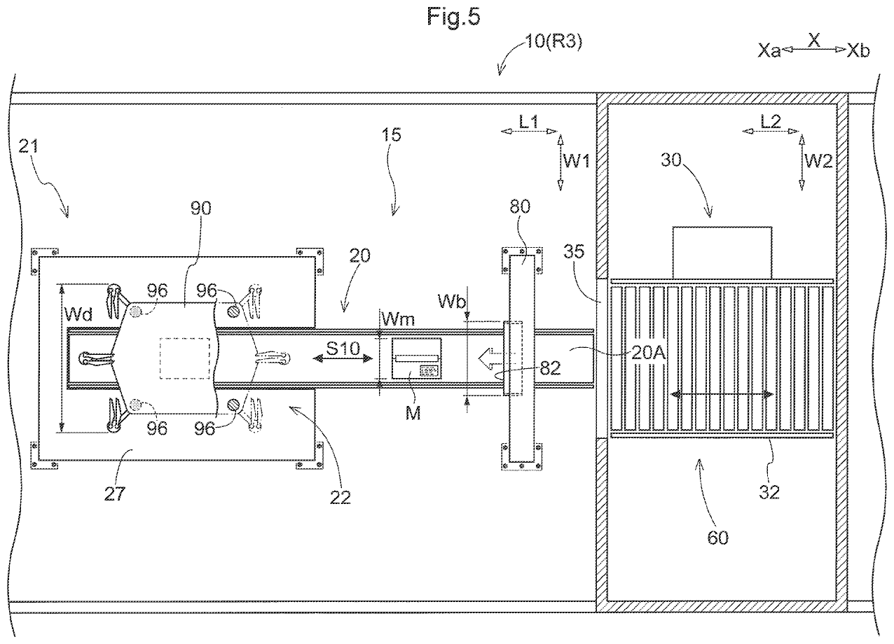

Hereinafter, a package receiving and storage apparatus 10 according to a second embodiment will be described with reference to the drawings. The present embodiment differs from the above first embodiment in that the package receiving and storage apparatus 10 does not include the package delivery section 23, the inclined section 25, the distribution transport device 26, the replenishment housing section 50, and the aircraft transport device 70, and that the external transport device 20 and the lifter 30 do not transport the unmanned aircraft 90. The following description focuses on differences from the above first embodiment. Aspects that are not specifically described are the same as those of the above first embodiment. FIG. 5 is an enlarged plan view showing the package receiving and storage apparatus 10 according to the second embodiment on the roof R3, and corresponds to FIG. 2.

In the present embodiment, a landing platform that is C-shaped in a vertical view is used as the support stand 27 that supports the unmanned aircraft 90 from below. One end of the external transport device 20 in the transport direction L1 is located at a central portion of the support stand 27 in a vertical view. The one end of the external transport device 20 also serves as the package receiving section 22. Accordingly, the package receiving section 22, which is one end of the external transport device 20, is located directly below the unmanned aircraft 90 that has landed on the support stand 27, which is a C-shaped landing platform. Also, the external transport device 20 is configured to transport the package M to the lifter 30 located on the other end side.

In the present embodiment, the gas spray device 80 is installed in such a manner as to spray the gas onto the package M located on the external transport device 20. Also, in the present embodiment, the gas spray device 80 emits the gas only during passage of the package M therethrough. In the illustrated example, the gas spray device 80 is shaped like a gate and includes the upper spray part 82, and the dimension Wb of the upper spray part 82 in the transport width direction W1 is larger than the dimension Wm of the package M, but is smaller than the dimension Wd of the unmanned aircraft 90.

### Third Embodiment

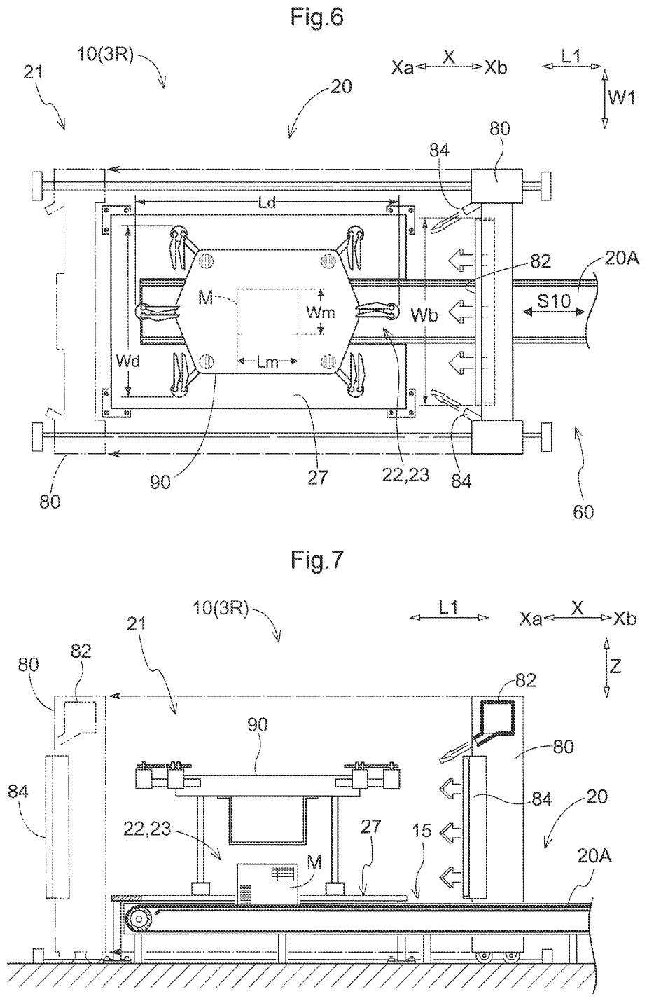

Hereinafter, a package receiving and storage apparatus 10 according to a third embodiment will be described with reference to the drawings. The present embodiment differs from the above second embodiment in that the gas spray device 80 moves in the transport direction L1. The following description focuses on differences from the above second embodiment. Aspects that are not specifically described are the same as those of the above second embodiment. In the present embodiment, the package receiving section 22 also serves as the package delivery section 23. FIG. 6 is an enlarged plan view showing the package receiving and storage apparatus 10 according to the third embodiment on the roof R3, and corresponds to FIG. 2. FIG. 7 is an enlarged side view showing the package receiving and storage apparatus 10 according to the third embodiment on the roof R3. In FIG. 7, the external transport device 20 and the gas spray device 80 are shown in a cross section.

In the present embodiment, the gas spray device 80 is installed on the package receiving section 22, and is configured to move in the transport direction L1, thereby spraying the gas onto the package M on the package receiving section 22 and the unmanned aircraft 90 that has landed on takeoff and landing section 21. In the present embodiment, the gas spray device 80 is configured to spray the gas at the support stand 27, which is the C-shaped landing platform, and the package receiving section 22 located at a central portion of the support stand 27, and spray the gas over a range wider than the dimension Wd of the unmanned aircraft 90 in the transport width direction W1. In the illustrated example, the gas spray device 80 is shaped like a gate and includes the upper spray part 82, and the dimension Wb of the upper spray part 82 in the transport width direction W1 is larger than the dimension Wm of the package M and the dimension Wd of the unmanned aircraft 90. Also, the gas spray device 80 includes, at column portions thereof, column spray parts 84 capable of spraying the gas onto side surfaces of the package M.

### Fourth Embodiment

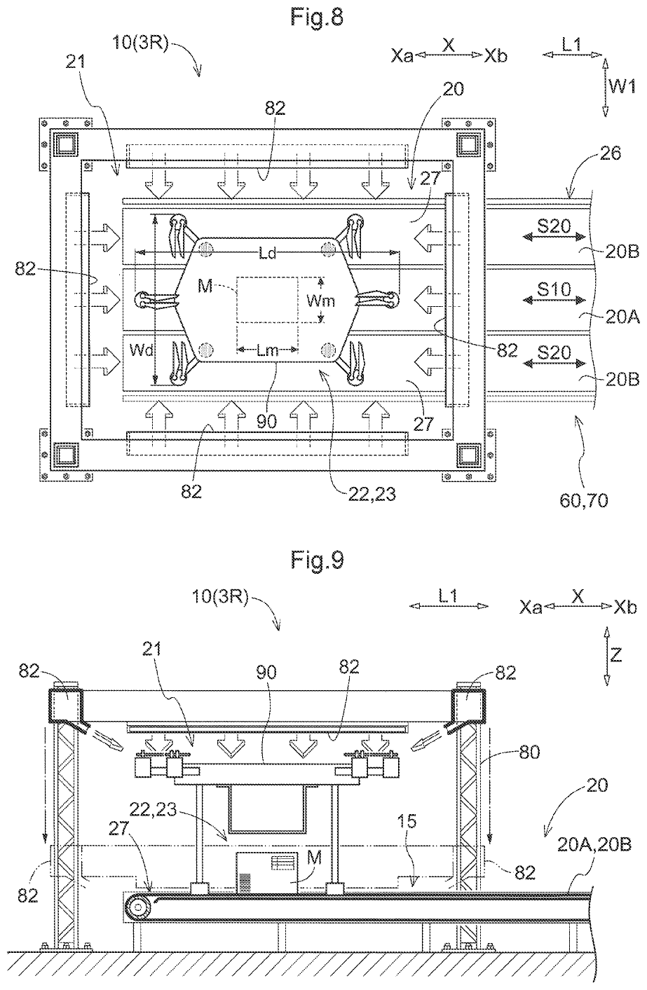

Hereinafter, a package receiving and storage apparatus 10 according to a fourth embodiment will be described with reference to the drawings. The present embodiment differs from the above first embodiment in that the gas spray device 80 moves in the vertical direction Z. The following description focuses on differences from the above first embodiment. Aspects that are not specifically described are the same as those of the above first embodiment. FIG. 8 is an enlarged plan view showing the package receiving and storage apparatus 10 according to the fourth embodiment on the roof R3, and corresponds to FIG. 2. FIG. 9 is an enlarged side view showing the package receiving and storage apparatus 10 according to the fourth embodiment on the roof R3. In FIG. 9, the external transport device 20 and the gas spray device 80 are shown in a cross section.

In the present embodiment, the gas spray device 80 is installed on the package receiving section 22, and moves in the vertical direction Z, thereby spraying the gas onto the package M on the package receiving section 22 and the unmanned aircraft 90 that has landed on the takeoff and landing section 21. In the present embodiment, the gas spray device 80 is included in the package receiving section 22, and is configured to spray the gas over a range wider than the dimension Wm of the package M in the transport width direction W1 and a dimension Lm of the package M in the transport direction L1. In the present embodiment, the gas spray device 80 is provided in the package receiving section 22 also serving as the takeoff and landing section 21, and is configured to spray the gas over a range wider than the dimension Wd of the unmanned aircraft 90 in the transport width direction W1 and the dimension Ld of the unmanned aircraft 90 in the transport direction L1. In the present embodiment, the gas spray device 80 includes upper spray parts 82 capable of moving in the vertical direction Z. In the illustrated example, the upper spray parts 82 each have a substantially rectangular shape in a vertical view. Preferably, the upper spray parts 82 are located below the upper spray parts 82 in the vertical direction Z during takeoff and landing of the unmanned aircraft 90, and the upper spray parts 82 are located above the unmanned aircraft 90 or the package M in the vertical direction Z when the unmanned aircraft 90 or the package M is transported toward the lifter 30.

### Other Embodiments

Next, other embodiments of the package receiving and storage apparatus 10 will be described.

(1) The above embodiments have each described an example in which the unmanned aircraft 90 is a rechargeable rotary-wing aircraft capable of vertical takeoff and landing. However, the present disclosure is not limited to such an example, and the unmanned aircraft 90 may be an electrically powered rotary-wing aircraft or fixed-wing aircraft including a fuel cell.

(2) The above embodiments have described, as an example, a configuration in which the transport device 15 includes the external transport device 20 that transports the package M at least in the horizontal direction, and the lifter 30 that transports the package M at least in the vertical direction Z, and the gas spray device 80 is configured to spray the gas onto the package M located on the external transport device 20. However, the present disclosure is not limited to such an example. For example, it is possible to adopt a configuration in which the transport device 15 does not include the external transport device 20 and the lifter 30, and the gas spray device 80 is installed in such a manner as to spray the gas onto the package M located on the package receiving section 22. Also, the gas spray device 80 may be installed in such a manner as to spray the gas onto the package M passing through the first entrance 35 provided on the roof R3, or the second entrances (retrieval sections 45) provided on each floor. It is also possible to adopt a configuration in which the transport device 15 includes an external transport device 20 that functions as the first transporter that transports the package M at least in the horizontal direction, and does not include the second transporter. It is also possible to adopt a configuration in which the transport device 15 includes a lifter 30 or a stacker crane that functions as the second transporter that transports the package M at least in the vertical direction Z, and does not include the first transporter.

(3) The above embodiments have described, as an example, a configuration in which the gas spray device 80 is disposed on the roof R3 of a building. However, the present disclosure is not limited to such an example. For example, the gas spray device 80 may be provided on the lifter 30 or a stacker crane that functions as the second transporter disposed inside the building. It is also possible to adopt a configuration in which a plurality of gas spray devices 80 are provided. Also, the gas spray device 80 may be configured to constantly emit the gas. Also, the column spray parts 84 may be provided in the gas spray devices 80 according to the first embodiment and the second embodiment.

(4) The above embodiments have described, as an example, a configuration in which the replenishment housing section 50 includes the wireless charging devices 53. However, the present disclosure is not limited to such an example. For example, the replenishment housing section 50 may include wired charging devices 53. Also, the replenishment housing section 50 may replenish the fuel cell of the unmanned aircraft 90, for example. In this case, the replenishment of energy to the unmanned aircraft 90 by the replenishment housing section 50 is performed through replenishment of a fuel serving as energy source (e.g., filling of a hydrogen storage container with hydrogen).

(5) The above embodiments have described, as an example, in which the package receiving and storage apparatus 10 is included in the building with two above-ground floors. However, the present disclosure is not limited to such an example. For example, the package receiving and storage apparatus 10 may be included in a multi-floor building that has a basement, or a plurality of buildings that share the package transport device 60 and the aircraft transport device 70. Also, a basement floor or a floor higher than the first above-ground floor may be the first floor R1 including the package entrance/exit port 41 and the unmanned aircraft entrance/exit port 51. Also, the package entrance/exit port 41 and the unmanned aircraft entrance/exit port 51 may be provided on floors different from each other.

(6) The above embodiments have described, as an example, a configuration in which, in the transport device 15, the package transport device 60 and the aircraft transport device 70 include the lifter 30, which is a shared second transporter. However, the present disclosure is not limited to such an example. For example, the package transport device 60 and the aircraft transport device 70 may each include a separate lifter 30. For example, it is possible to adopt a configuration in which the package receiving and storage apparatus 10 is included in a building with only one above-ground floor, and the package transport device 60 is a stacker crane that transports the package M to each of a plurality of rows of a storage rack that functions as the storage section 40. It is also possible to adopt a configuration in which the aircraft transport device 70 is a stacker crane that transports the unmanned aircraft 90 to each of a plurality of rows of a housing rack that functions as the replenishment housing section 50. The package transport device 60 and the aircraft transport device 70 may each include a separate stacker crane, or may include a shared stacker crane. The retrieval sections 45 may be retrieval ports for a stacker crane, including, for example, retrieval racks and retrieval conveyors.

(7) The above embodiments have described, as an example, a configuration in which the storage section 40 is adjacent to the lifter 30 on the first side Xa in the specific direction, and the replenishment housing section 50 is adjacent to the lifter 30 on the second side X13 in the specific direction. However, the present disclosure is not limited to such an example. For example, both the storage section 40 and the replenishment housing section 50 may be spaced away from the lifter 30 on the second side X13 in the specific direction relative to the lifter 30. Also, the storage transport device 61 and the housing transport device 71 may be automated guided vehicles that travel between the storage section 40, the replenishment housing section 50, and the lifter 30.

(8) The above embodiments have described, as an example, a configuration in which the external transport device 20 is provided on the roof R3, and also serves as the takeoff and landing section 21. However, the present disclosure is not limited to such an example. For example, the external transport device 20 may be disposed downstream of the takeoff and landing section 21. For example, the external transport device 20 need not overlap the storage section 40 in a vertical view. For example, the takeoff and landing section 21 may be in the form of a platform, or may be in the form of a hanger in which the unmanned aircraft 90 is hooked and hung. For example, a takeoff portion and a landing portion of the takeoff and landing section 21 may be provided in separate locations, or may each be provided in a plurality of locations.

(9) The above embodiments have described, as an example, a configuration in which the external transport device 20 is provided on the roof R3, and also serves as the package receiving section 22 and the package delivery section 23. However, the present disclosure is not limited to such an example. For example, the package receiving section 22 and the package delivery section 23 may be provided in separate locations. For example, the package receiving section 22 may be provided on the first floor R1 rather than the roof R3. As another example, the package receiving section 22 and the package delivery section 23 may each be provided in a plurality of locations. As another example, the external transport device 20 also serving as the takeoff and landing section 21 may be provided indoors.

(10) The above embodiments have described, as an example, a configuration in which the external transport device 20 includes the package delivery device 24 also serving as the package receiving device. However, the present disclosure is not limited to such an example. For example, a configuration is possible in which the package receiving section 22 and the package delivery section 23 are not provided with the package delivery device 24, and an operator performs a package receiving operation and a package delivery operation. Also, the package receiving section 22 may be a placement table or a chute that does not include any package receiving device.

(11) The above embodiments have described, as an example, a configuration in which the gas spray device 80 sprays the gas over a range wider than the dimension Wm of the package M in the transport width direction W1. However, the present disclosure is not limited to such an example. For example, it is possible to adopt a configuration in which the gas spray device 80 sprays the gas over a range wider than the dimension Lm of the package M in the transport direction L1.

(12) Note that the configurations disclosed in the embodiments described above are applicable in combination with configurations disclosed in other embodiments as long as no inconsistency arises. With regard to the other configurations as well, the embodiments disclosed herein are illustrative in all respects. Therefore, various modifications and alterations may be made as appropriate without departing from the gist of the present disclosure.

### Outline of the Embodiment

Hereinafter, the package receiving and storage apparatus described above will be described.

A package receiving and storage apparatus according to the present disclosure includes: a takeoff and landing section at which an unmanned aircraft is capable of taking off and landing; a package receiving section configured to receive a first package from the unmanned aircraft; a storage section configured to store a plurality of packages including the first package; a retrieval section at which the first package stored in the storage section is retrievable; a transport device configured to transport the first package between the package receiving section, the storage section, and the retrieval section; and a gas spray device installed at the package receiving section or in a package transport path extending from the package receiving section to the storage section, and configured to spray a gas onto the first package to remove foreign matter adhering to the first package.

With this configuration, it is possible to remove foreign matter adhering to the package received from the unmanned aircraft before storing the package in the storage section. Accordingly, it is possible to easily avoid, for example, a situation where foreign matter separated from the package inside the storage section accumulates as waste matter, dirtiness, or the like inside the storage section. Note that such foreign matter is assumed to be, for example, water droplets resulting from rain and snow, dust, fallen leaves, and the like. Also, this configuration can reduce the possibility that a package having foreign matter adhering thereto arrives at the retrieval section.

As an aspect, it is preferable that the transport device includes a first transporter configured to transport the first package at least in a horizontal direction, and the gas spray device is installed in such a manner as to spray the gas onto the first package located on the first transporter.

With this configuration, the gas spray device sprays the gas onto the package located on the first transporter, and it is thus possible to simplify the gas spray device, and also to quickly transport the package received from the unmanned aircraft.

As an aspect, it is preferable that the takeoff and landing section includes a support stand configured to support the unmanned aircraft from below, the storage section is disposed lower than the support stand, the transport device includes: the first transporter disposed higher than the storage section; and a second transporter configured to transport the first package at least in a vertical direction, and the first transporter also serves as the package receiving section, and transports the first package to the second transporter from a position directly below the unmanned aircraft supported by the support stand.

With this configuration, it is possible to utilize a space located below the takeoff and landing section to store a plurality of packages. With this configuration, the first transporter also serves as the package receiving section, and it is thus possible to simplify the configuration of the apparatus, and also to quickly transport the package received from the unmanned aircraft.

As an aspect, it is preferable that the gas spray device sprays the gas at a target position on a transport path of the first transporter, and sprays the gas over a range wider than a dimension of the first package in a transport width direction extending orthogonal, in a vertical view, to a transport direction extending along the transport path of the first transporter.

With this configuration, it is possible to utilize movement of the package by the first transporter to spray the gas onto the entire package.

As an aspect, it is preferable that the takeoff and landing section and the first transporter overlap the storage section in the vertical view.

This configuration makes it possible to easily minimize the placement area of the package receiving and storage apparatus in a plan view, while ensuring that a sufficient number of packages can be stored.

As an aspect, it is preferable that the gas spray device further sprays the gas onto the unmanned aircraft landed on the takeoff and landing section.

With this configuration, it is also possible to remove foreign matter adhering to the unmanned aircraft.

As an aspect, it is preferable that the storage section is disposed inside a building, and the takeoff and landing section, the package receiving section, and the gas spray device are disposed on a roof of the building.

With this configuration, it is possible to provide a configuration that can facilitate takeoff and landing of the unmanned aircraft. Also, it is possible to easily provide a configuration in which foreign matter that has been blown away by the gas spray device are less likely to enter the inside of the building. cm 1. A package receiving and storage apparatus comprising:
a takeoff and landing section at which an unmanned aircraft is capable of taking off and landing;
a package receiving section configured to receive a first package from the unmanned aircraft;
a storage section configured to store a plurality of packages comprising the first package;
a retrieval section at which the first package stored in the storage section is retrievable;
a transport device configured to transport the first package between the package receiving section, the storage section, and the retrieval section; and
a gas spray device installed at the package receiving section or in a package transport path extending from the package receiving section to the storage section, and configured to spray a gas onto the first package to remove foreign matter adhering to the first package.

2. The package receiving and storage apparatus according to claim 1,
wherein the transport device comprises a first transporter configured to transport the first package at least in a horizontal direction, and
wherein the gas spray device is installed in such a manner as to spray the gas onto the first package located on the first transporter.

3. The package receiving and storage apparatus according to claim 2, wherein:
the takeoff and landing section comprises a support stand configured to support the unmanned aircraft from below,
the storage section is disposed lower than the support stand,
the transport device comprises:
the first transporter disposed higher than the storage section; and
a second transporter configured to transport the first package at least in a vertical direction, and
the first transporter also serves as the package receiving section, and transports the first package to the second transporter from a position directly below the unmanned aircraft supported by the support stand.

4. The package receiving and storage apparatus according to claim 2,
wherein the gas spray device sprays the gas at a target position on a transport path of the first transporter, and sprays the gas over a range wider than a dimension of the first package in a transport width direction extending orthogonal, in a vertical view, to a transport direction extending along the transport path of the first transporter.

5. The package receiving and storage apparatus according to claim 2,
wherein the takeoff and landing section and the first transporter overlap the storage section in the vertical view.

6. The package receiving and storage apparatus according to claim 1,
wherein the gas spray device further sprays the gas onto the unmanned aircraft landed on the takeoff and landing section.

7. The package receiving and storage apparatus according to claim 1,
wherein the storage section is disposed inside a building, and
wherein the takeoff and landing section, the package receiving section, and the gas spray device are disposed on a roof of the building.
